(19) United States
(12) Patent Application Publication
(10) Pub. No.: US 2015/0025061 A1
(43) Pub. Date: Jan. 22, 2015

(54) GABAA RECEPTOR ANTAGONISTS AFFECTING GANGLION CELL FUNCTION AND VISUAL ACUITY

(71) Applicant: Allergan, Inc., Irvine, CA (US)

(72) Inventor: Movses H. Karakossian, Foothill Ranch, CA (US)

(21) Appl. No.: 14/327,805

(22) Filed: Jul. 10, 2014

Related U.S. Application Data
(60) Provisional application No. 61/846,924, filed on Jul. 16, 2013.

(51) Int. Cl. A61K 31/50; A61K 31/4741; A61K 31/365; A61K 31/53; A61K 31/5025; A61K 31/551; A61K 31/427; A61K 31/55; A61K 31/5517

(52) U.S. Cl. CPC A61K 31/50; A61K 31/55; A61K 31/4741; A61K 31/365; A61K 31/5517; A61K 31/5025; A61K 31/551; A61K 31/427; A61K 31/53
USPC 514/211.09; 514/247; 514/291; 514/468; 514/219; 514/248; 514/220; 514/365; 514/243

(57) ABSTRACT

The present invention is directed to a method of enhancing visual acuity in a subject, comprising intravitreally administering to the subject in need of such enhancement, a therapeutically effective amount of an extrasynaptic GABA_{A} receptor antagonist. The present invention is also directed to an ocular implant comprising a therapeutically effective amount of the extrasynaptic GABA_{A} receptor antagonist.

### CROSS REFERENCE TO RELATED APPLICATIONS

This application claims the benefit of U.S. Provisional Application Ser. No. 61/846,924 filed on Jul. 16, 2013 of which is incorporated by reference herein in its entirety.

### FIELD OF THE INVENTION

The present invention is directed to methods of enhancing visual function and for treating ocular conditions resulting from low or poor visual function by administration of a GABA_{A} receptor antagonist.

### BACKGROUND OF THE INVENTION

Gamma-amino butyric acid (GABA) is a major inhibitory neurotransmitter in the central nervous system which include the brain, spinal cord and the retina (for review see: Macdonald and Olsen, 1994). GABA releasing neurons are diverse and control the activity of neuronal circuits by imposing inhibition on their postsynaptic counterparts. Receptors that bind GABA are found in almost all neuronal types and represent a diverse array of receptor types (Mody and Pearce, 2004). Ionotropic GABA receptors (GABAaRs) are ligand activated chloride channels which are heteropentamer members of the Cys-loop ligand-gated ion channel superfamily permeable to chlorine ions that upon opening hyperpolarize neurons in the adult nervous system (Bernard et al., 1998). They are composed of two α-, two β-, and one γ-subunits. Nineteen total subunits, i.e., α1-6, β1-3, γ1-3, δ, ε, τ, π and ρ1-3, that could arrange in enormous number of theoretical pentameric combinations are identified to date (for review see: Olsen and Sieghart, 2009).

Following the release of GABA from the presynaptic terminal, GABA binds to GABAaRs that are located on the postsynaptic membrane of the synaptic specializations hence these receptors are termed synaptic GABAa receptors (Bernard et al., 1998). Following the release of GABA from the presynaptic terminal, GABA spills over from the synapse into perisyanptic and extrasynaptic sites where it binds to a different subtype of GABAa receptors termed extrasynaptic GABAa receptors (Somogyi et al., 1989), and gives rise to a tonic GABA-mediated current (otis et al., 1991). Tonic inhibition is distinct from the transient activation of synaptic GABAaRs leading to classical inhibitory postsynaptic currents (phasic inhibition). The initial finding in cerebellar granule cells (Brickley et al., 1996; Wall and Usowicz, 1997; Nusser et al., 1998; Brickley et al., 2001; Hamann et al., 2002) was followed by subsequent discoveries in, among others, the dentate gyrus and hippocampus (Bai et al., 2001; Nusser and Mody, 2002; Semyanov et al., 2003; Wei et al., 2003; Caraiscos et al., 2004a,b; Scimemi et al., 2005; Glykys et al., 2007), neocortex (Drasbek and Jensen, 2006; Yamada et al., 2007; Krook-Magnuson et al., 2008), thalamus (Belelli et al., 2005; Cope et al., 2005; Jia et al., 2005), striatum (Ade et al., 2008; Janssen et al., 2009), hypothalamus (Park et al., 2006, 2007), spinal cord (Takahashi et al., 2006; Wang et al., 2008), and retina (Wang et al., 2007). The occurrence of tonic GABAa inhibition coincides with the expression of relatively rare receptor subunits, particularly the α4, α6, and δ subunits, and as a general rule-of-thumb, δ subunit-containing receptors are extrasynaptic, but not all extrasynaptic GABAaRs contain δ subunits. In comparison, the ubiquitous γ2 subunit is a major component of synaptic GABAaRs and drives receptor clustering at the synapse (Essrich et al., 1998). The presence of the δ subunit in recombinant receptors conveys properties ideally suited to generating tonic inhibition, namely activation by low concentrations of GABA, such as may be found in the extracellular space and reduced desensitization (Saxena and Macdonald, 1994; Haas and Macdonald, 1999; Bianchi and Macdonald, 2002; Brown et al., 2002). In addition to δ subunit-containing receptors, other subunit compositions are also capable of generating tonic GABA conductance, namely α5 containing receptors (Glykys and Mody, 2006, 2007).

GABAa receptors have been implicated in disorders such as: epilepsy, sleep disorders, stress and psychiatric disorders, alcoholism, cognitive disorders (for review see: Brickley and Mody, 2012).

US 2006/0264508 A1 refers to methods and compositions for controlling postnatal ocular growth and the development of ocular errors in the maturing eye of a subject, comprising altering the refraction and/or growth of the maturing eye of a subject by administering to the eye a therapeutically effective amount of at least one GABA drug or a compound, including agonists or antagonists.

### BIBLIOGRAPHY

Ade K K, Janssen M J, Ortinski P I, Vicini S (2008) Differential tonic GABA conductances in striatal medium spiny neurons. J Neurosci 28:1185-1197.
Bai D, Zhu G, Pennefather P, Jackson M F, MacDonald J F, Orser B A (2001) Distinct functional and pharmacological properties of tonic and quantal inhibitory postsynaptic currents mediated by gamma-aminobutyric acid(A) receptors in hippocampal neurons. Mol Pharmacol 59:814-824.
Barnard E A, Skolnick P, Olsen R W, Mohler H, Sieghart W, Biggio G, Braestrup C, Bateson A N, Langer S Z (1998) International Union of Pharmacology. X V. Subtypes of gamma-aminobutyric acidA receptors: classification on the basis of subunit structure and receptor function. Pharmacol Rev 50:291-313.
Belelli D, Peden D R, Rosahl T W, Wafford K A, Lambert J J (2005) Extrasynaptic GABAA receptors of thalamocortical neurons: a molecular target for hypnotics. J Neurosci 25:11513-11520.
Bianchi M T, Haas K F, Macdonald R L (2002) Alpha1 and alpha6 subunits specify distinct desensitization, deactivation and neurosteroid modulation of GABA(A) receptors containing the delta subunit. Neuropharmacology 43:492-502.
Brickley S G, Cull-Candy S G, Farrant M (1996) Development of a tonic form of synaptic inhibition in rat cerebellar granule cells resulting from persistent activation of GABAA receptors. J Physiol 497 (Pt 3):753-759.
Brickley S G, Farrant M, Swanson G T, Cull-Candy S G (2001) CNQX increases GABA-mediated synaptic transmission in the cerebellum by an AMPA/kainate receptor-independent mechanism. Neuropharmacology 41:730-736.
Brickley S G, Mody I (2012) Extrasynaptic GABA(A) receptors: their function in the CNS and implications for disease. Neuron 73:23-34.
Brown N, Kerby J, Bonnert T P, Whiting P J, Wafford K A (2002) Pharmacological characterization of a novel cell line expressing human alpha(4)beta(3)delta GABA(A) receptors. Br J Pharmacol 136:965-974.
Caraiscos V B, Elliott E M, You-Ten K E, Cheng V Y, Belelli D, Newell J G, Jackson M F, Lambert J J, Rosahl T W, Wafford K A, MacDonald J F, Orser B A (2004a) Tonic inhibition in mouse hippocampal CA1 pyramidal neurons is mediated by alpha5 subunit-containing gamma-aminobutyric acid type A receptors. Proc Natl Acad Sci USA 101:3662-3667.
Caraiscos V B, Newell J G, You-Ten K E, Elliott E M, Rosahl T W, Wafford K A, MacDonald J F, Orser B A (2004b) Selective enhancement of tonic GABAergic inhibition in murine hippocampal neurons by low concentrations of the volatile anesthetic isoflurane. J Neurosci 24:8454-8458.
Cope D W, Hughes S W, Crunelli V (2005) GABAA receptor-mediated tonic inhibition in thalamic neurons. J Neurosci 25:11553-11563.
Douglas R M, Alam N M, Silver B D, McGill T J, Tschetter W W, Prusky G T (2005) Independent visual threshold measurements in the two eyes of freely moving rats and mice using a virtual-reality optokinetic system. Vis Neurosci 22:677-684.
Drasbek K R, Jensen K (2006) THIP, a hypnotic and antinociceptive drug, enhances an extrasynaptic GABAA receptor-mediated conductance in mouse neocortex. Cereb Cortex 16:1134-1141.
Essrich C, Lorez M, Benson J A, Fritschy J M, Luscher B (1998) Postsynaptic clustering of major GABAA receptor subtypes requires the gamma 2 subunit and gephyrin. Nat Neurosci 1:563-571.
Glykys J, Mody I (2006) Hippocampal network hyperactivity after selective reduction of tonic inhibition in GABA A receptor alpha5 subunit-deficient mice. J Neurophysiol 95:2796-2807.
Glykys J, Mody I (2007) The main source of ambient GABA responsible for tonic inhibition in the mouse hippocampus. J Physiol 582:1163-1178.
Guire E S, Lickey M E, Gordon B (1999) Critical period for the monocular deprivation effect in rats: assessment with sweep visually evoked potentials. J Neurophysiol 81:121-128.
Haas K F, Macdonald R L (1999) GABAA receptor subunit gamma2 and delta subtypes confer unique kinetic properties on recombinant GABAA receptor currents in mouse fibroblasts. J Physiol 514 (Pt 1):27-45.
Hamann M, Rossi D J, Attwell D (2002) Tonic and spillover inhibition of granule cells control information flow through cerebellar cortex. Neuron 33:625-633.
Janssen M J, Ade K K, Fu Z, Vicini S (2009) Dopamine modulation of GABA tonic conductance in striatal output neurons. J Neurosci 29:5116-5126.
Jia F, Pignataro L, Schofield C M, Yue M, Harrison N L, Goldstein P A (2005) An extrasynaptic GABAA receptor mediates tonic inhibition in thalamic V B neurons. J Neurophysiol 94:4491-4501.
Krook-Magnuson E I, Li P, Paluszkiewicz S M, Huntsman M M (2008) Tonically active inhibition selectively controls feedforward circuits in mouse barrel cortex. J Neurophysiol 100:932-944.
Macdonald R L, Olsen R W (1994) GABAA receptor channels. Annu Rev Neurosci 17:569-602.
Mody I, Pearce R A (2004) Diversity of inhibitory neurotransmission through GABA(A) receptors. Trends Neurosci 27:569-575.
Norcia A M, Tyler C W (1985) Spatial frequency sweep VEP: visual acuity during the first year of life. Vision Res 25:1399-1408.
Nusser Z, Mody I (2002) Selective modulation of tonic and phasic inhibitions in dentate gyrus granule cells. J Neurophysiol 87:2624-2628.
Nusser Z, Sieghart W, Somogyi P (1998) Segregation of different GABAA receptors to synaptic and extrasynaptic membranes of cerebellar granule cells. J Neurosci 18:1693-1703.
Olsen R W, Sieghart W (2009) GABA A receptors: subtypes provide diversity of function and pharmacology. Neuropharmacology 56:141-148.
Otis T S, Staley K J, Mody I (1991) Perpetual inhibitory activity in mammalian brain slices generated by spontaneous GABA release. Brain Res 545:142-150.
Park J B, Skalska S, Son S, Stern J E (2007) Dual GABAA receptor-mediated inhibition in rat presympathetic paraventricular nucleus neurons. J Physiol 582:539-551.
Park J B, Skalska S, Stern J E (2006) Characterization of a novel tonic gamma-aminobutyric acidA receptor-mediated inhibition in magnocellular neurosecretory neurons and its modulation by glia. Endocrinology 147:3746-3760.
Prusky G T, Alam N M, Beekman S, Douglas R M (2004) Rapid quantification of adult and developing mouse spatial vision using a virtual optomotor system. Invest Ophthalmol Vis Sci 45:4611-4616.
Ridder W H, 3rd (2004) Methods of visual acuity determination with the spatial frequency sweep visual evoked potential. Doc Ophthalmol 109:239-247.
Saxena N C, Macdonald R L (1994) Assembly of GABAA receptor subunits: role of the delta subunit. J Neurosci 14:7077-7086.
Scimemi A, Semyanov A, Sperk G, Kullmann D M, Walker M C (2005) Multiple and plastic receptors mediate tonic GABAA receptor currents in the hippocampus. J Neurosci 25:10016-10024.
Semyanov A (2003) Cell type specificity of GABA(A) receptor mediated signaling in the hippocampus. Curr Drug Targets CNS Neurol Disord 2:240-247.
Somogyi P, Takagi H, Richards J G, Mohler H (1989) Subcellular localization of benzodiazepine/GABAA receptors in the cerebellum of rat, cat, and monkey using monoclonal antibodies. J Neurosci 9:2197-2209.
Takahashi A, Mashimo T, Uchida I (2006) GABAergic tonic inhibition of substantia gelatinosa neurons in mouse spinal cord. Neuroreport 17:1331-1335.
Wall M J, Usowicz M M (1997) Development of action potential-dependent and independent spontaneous GABAA receptor-mediated currents in granule cells of postnatal rat cerebellum. Eur J Neurosci 9:533-548.
Wang C T, Blankenship A G, Anishchenko A, Elstrott J, Fikhman M, Nakanishi S, Feller M B (2007) GABA(A) receptor-mediated signaling alters the structure of spontaneous activity in the developing retina. J Neurosci 27:9130-9140.
Wang L, Spary E, Deuchars J, Deuchars S A (2008) Tonic GABAergic inhibition of sympathetic preganglionic neurons: a novel substrate for sympathetic control. J Neurosci 28:12445-12452.
Yamada J, Furukawa T, Ueno S, Yamamoto S, Fukuda A (2007) Molecular basis for the GABAA receptor-mediated tonic inhibition in rat somatosensory cortex. Cereb Cortex 17:1782-1787.

### SUMMARY OF THE INVENTION

The present invention provides a method of enhancing visual function in a subject, comprising intravitreally administering to the subject in need of such enhancement, a therapeutically effective amount of a compound that is an extrasynaptic GABA_{A} receptor antagonist.

The present invention also provides a method of treating an ocular condition resulting from low/poor visual function in a subject, comprising intravitreally administering to said subject in need of such treatment, a therapeutically effective amount of a compound that is an extrasynaptic GABA_{A} receptor antagonist.

The present invention also provides an ocular implant comprising a therapeutically effective amount of a compound that is an extrasynaptic GABA_{A} receptor antagonist.

### BRIEF DESCRIPTION OF THE DRAWINGS

FIG. 1 shows that in the middle of the retina there is a small pit, the fovea, with which we see sharply. Only a few millimeters from the fovea (arrows) the visual acuity is 20/200 (6/60 or 0.1) even in a normal person.

FIG. 2 shows the visual pathways from the eyes to the visual cortex. Note that there are also connections to the central parts of the brain. Note also that in the optic radiation, the pathway from the LGN (lateral geniculate nucleus) to the primary visual cortex there are marked arrows in the direction from the primary visual cortex to the LGN. Actually, there are some ten times more fibres bringing information from the primary visual cortex to the LGN than in the opposite direction. From the primary visual cortex information flows “backwards” also to the superior colliculus (SC).

FIG. 3 illustrates visual field. 3A shows visual field of both eyes. 3B shows that the central part of the visual field (white area) is seen by both eyes.

FIG. 4 illustrates contrast sensitivity. 4A shows the contrast sensitivity curve. 4B shows visual information at different contrasts in different sizes. Note that large numbers are visible at a fainter contrast than smaller numbers.

FIG. 5 illustrates eye muscles seen from above. The left outer muscle has developed palsy, the left eye turns inward.

FIG. 6 shows GABA levels in the vitreous humor under normal and excitotoxic conditions during day and night. GABA levels were analyzed using the LCMS method. There was a significant difference in GABA levels under control and excitotoxic (NMDA) conditions (N=4, P<0.05). However there was no significant diurnal effect on GABA levels under both conditions (N=4, P>0.05). High vitreal GABA levels (˜2-3 uM) under control Conditions suggest tonic inhibition of cells expressing extrasynaptic GABAa receptors.

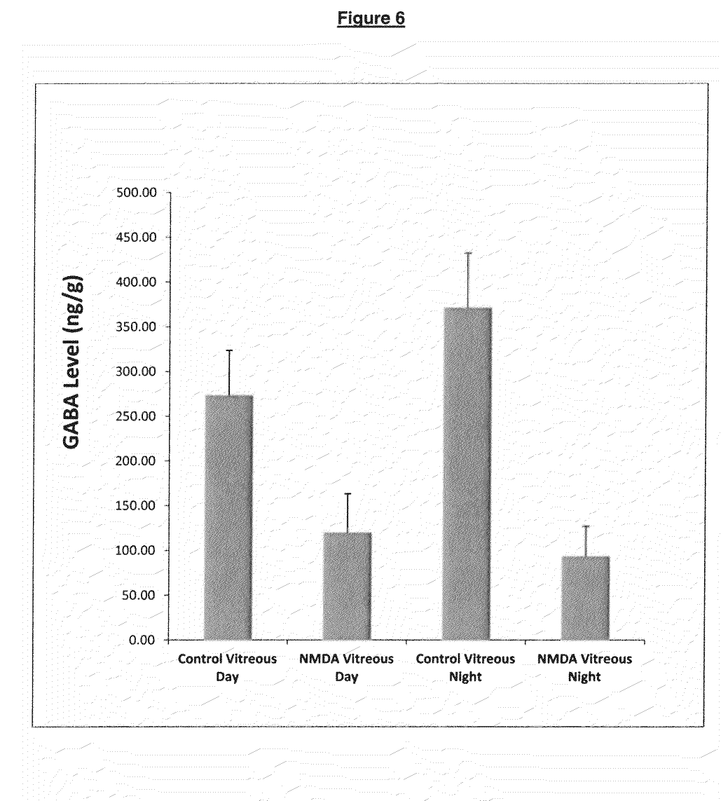

FIG. 7 shows the effects of intravitreal SR95531 on visual acuity measured by the rabit optomotor test. Following intravitreal injection of 5 uM SR95531 and Saline, visual acuity was attenuated in both eyes to an injection artifact. Following recovery from the injection, the eye treat with SR95531, visual acuity significantly improved at day 1 and 2 post injection (* P<0.05, N=4).

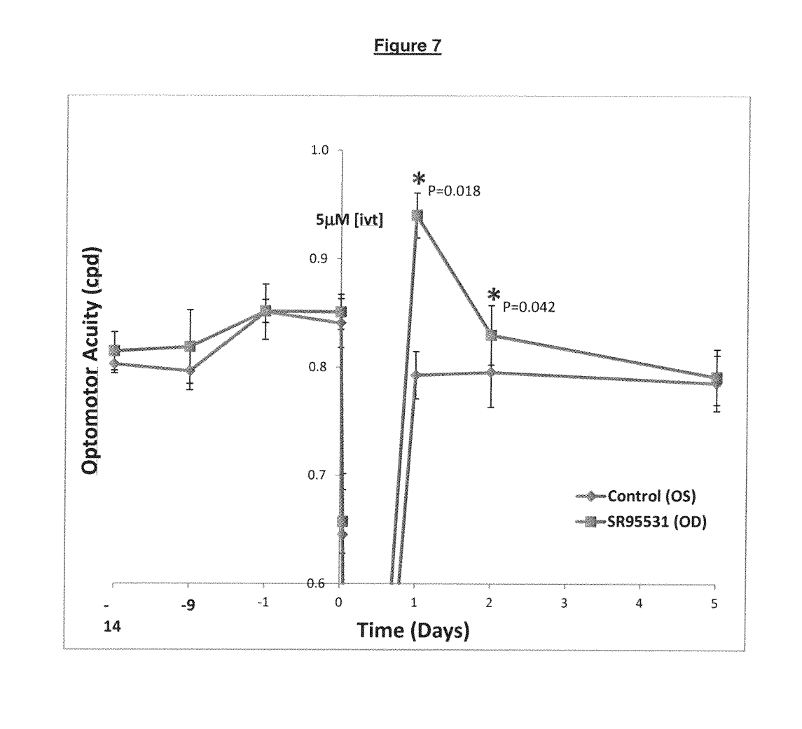

FIG. 8 shows the effects of intravitreal SR95531 on visual acuity measured by sweep vision evoked potential (VEP; see experimental methods for further details). Following intravitreal injection of different doses of SR95531 and Saline in the contralateral control eye, visual acuity was measured using sweep VEP method. At 1 uM SR95531, the effect was small but not statistically significant. At SuM, 15 uM and 50 uM, there was significant enhancement in visual acuity at 24 hours. The 50 uM dose of SR95531 resulted a delay in the enhancement of visual acuity suggesting that at high doses synaptic receptors are also targeted and as the dose dropped the enhancement in visual acuity was apparent (* P<0.05, N=4).

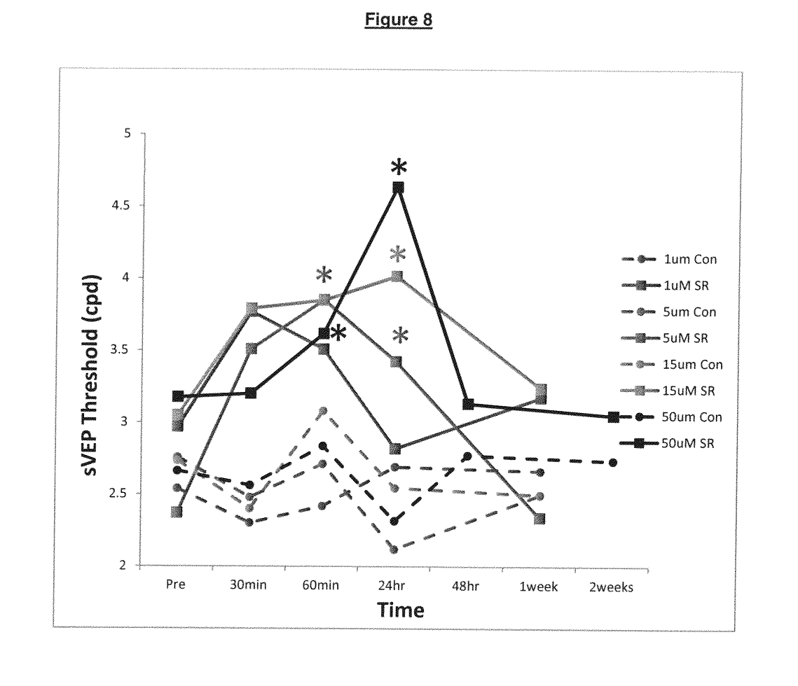

FIG. 9 shows the pharmacokinetic profile of SR95531 at different time-points in Dutch-belted rabbits. Following intravitreal dosing with 15 uM (50 uL of 360 uM) of SR95531 into both eyes, the animals were euthanized and different tissues were collected for pharmacokinetic analysis. The data shows the persistence of SR95531 in ocular tissues following the injection with maintained levels in the retina and choroid up to 1 week after injection. (N=4 eyes/time-point, except for the 1 and 4 hour time-points when N=3 due to outliers).

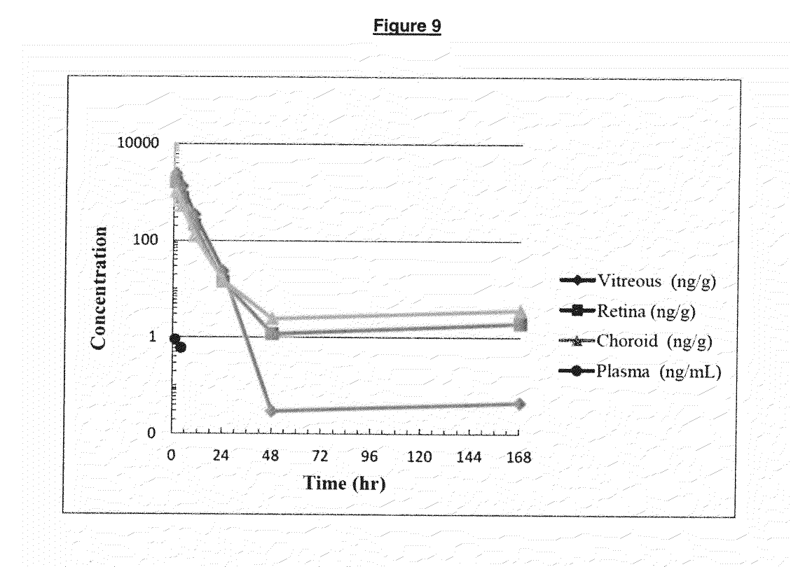

### DETAILED DESCRIPTION OF THE INVENTION

### Embodiments of the Invention

Vision is composed of many simultaneous functions. If vision is normal, seeing is so effortless that we do not notice the different visual functions.

The different components of the visual image are: forms, colors and movement. Thus we have form perception, color perception and motion perception.

We see both during the day light and during very dim light. In day light, photopic vision, we perceive colors because of function of the cone cells; in very dim light, scotopic vision, we see only shades of gray, since rod cells respond only to luminance differences. In twilight, when both rod and cone cells function, we have mesopic vision.

Vision is measured with many different tests, such as tests for visual acuity, visual field, contrast sensitivity, color vision, visual adaptation to different luminance levels, binocular vision and stereoscopic vision.

The term “visual function” as used herein includes all of the above, namely visual acuity, visual field, contrast sensitivity, color vision, visual adaptation to different luminance levels, binocular vision and three dimensional (stereoscopic) vision.

In another embodiment of the present invention, the visual function is visual acuity.

A good article on different visual functions is available on the web at http://www.lea-test.fi/en/eyes/visfunct.html.

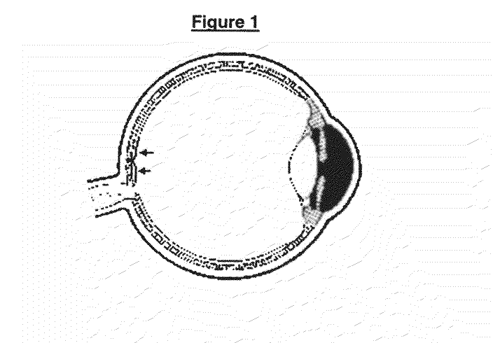

“Visual acuity” is measured with visual acuity charts at distance and at near. The test measures what is the smallest letter, number or picture size that the patient still sees correctly. Visual acuity is good only in the very middle of the retina. See FIG. 1.

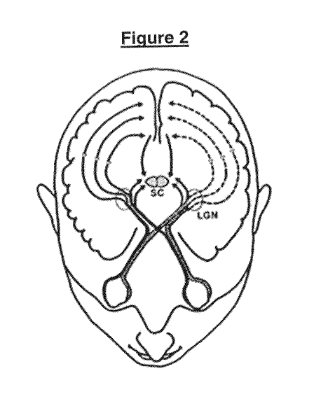

When a person with normal vision looks straight forward without moving the eyes, (s)he sees also on both sides. The area visible at once, without moving the eyes, is called “visual field”. Nerve fibres from both eyes are divided so that fibers from the right half of both eyes reach the right half of the brain and fibers from the left half of both eyes the left half of the brain. See FIG. 2.

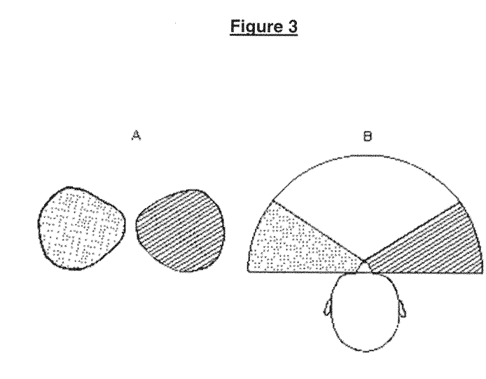

Visual information coming from both eyes is fused in the visual cortex in the back of the brain. The central part of the visual field is seen by both eyes (FIG. 3). On both sides of this central, binocular field there are half moon formed parts of visual field that are seen by only one eye. See FIG. 3.

We use our peripheral or side vision when moving around. The most central part of the visual field is used in sustained near work, e.g., reading. When the visual field is measured with the clinical instruments these instruments measure what the weakest light is that the eye still can see in different parts of the visual field. A measurement like this gives valuable information on diseases of the visual pathways related to glaucoma or neurologic diseases. It does not give information on how the person sees forms or perceives movement in the different parts of the visual field.

The visual field can change in many ways. Therefore it is often difficult to understand how a visually impaired person sees. If the side parts of the visual field function poorly the person may need to use a white cane in order to move around safely, but (s)he may be able to read without glasses. On the other hand, if the side parts function well and the central field functions poorly, the person may walk like a normally sighted person, but may be able to read only the headings of a newspaper.

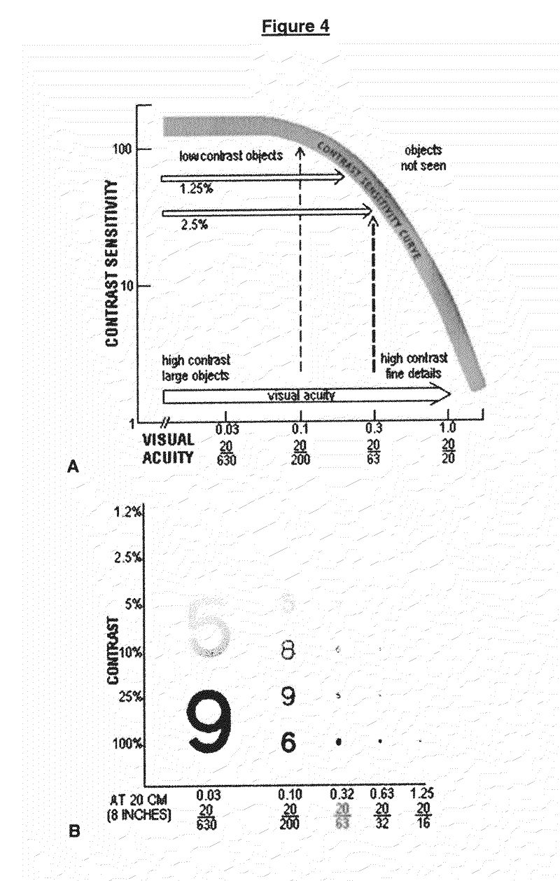

“Contrast sensitivity” can be depicted, for example, by a curve (See FIG. 4A). Under the curve there are the objects that we can see, above and to the right of the slope of the curve is the visual information that we cannot see. Contrast sensitivity can be measured using striped patterns, gratings, or symbols at different contrast levels.

When we measure hearing, an audiogram depicts which are the weakest tones at different frequencies that we still can hear. The measurements are made at low, intermediate and high frequencies. When we measure contrast sensitivity we measure what is the faintest grating or symbol still visible when the symbols are large, medium size or small (FIG. 4B).

If a visually impaired person has poor contrast sensitivity (s)he cannot see small contrast differences between adjacent surfaces. Everything becomes flat. It is difficult to perceive facial features and expressions. Text in the newspapers seems to have less contrast than before and it is difficult to recognize the edge of the pavement and the stairs.

Contrast sensitivity decreases in several common diseases, diabetes, glaucoma, cataract and diseases of the optic nerve.

Visual adaptation to different luminance levels: A normally sighted person can read by one candle's light and (s)he can read in bright sun light. The difference in the amount of light present in these two situations is million times. The normal person can adapt his/her vision to function at the different luminance levels.

The rod cells of the retina see best in twilight. If they do not function, the person is night blind. Night blindness is the first symptom that develops in many retinal diseases. First the child with a retinal disease starts to see in dim light after an abnormally long waiting. Therefore (s)he will have difficulties in finding his/her clothing in a closet or in a drawer if there is no extra illumination directed into these places. Later (s)he loses night vision completely, even when waiting for a long time (s)he does not start to see in the dark. Changes in visual adaptation time can be easily detected with the CONE Adaptation Test.

Photophobia and delayed adaptation to bright light are often additional symptoms of abnormal visual adaptation. When normally sighted persons enter from a darker room into a bright light, they also see very little for a second, sometimes it even hurts their eyes. They are dazzled. A visually impaired person may be dazzled for a long time. It is possible to decrease the problem by using absorptive glasses and a hat with wide brim or a visor.

Color vision: There are three different types of the retinal cone cells: some cells are most sensitive to red light, other to green light and the third type is most sensitive to blue light. Also the “normally sighted” individuals may have minor difficulties with color perception. It is often called color blindness but the term is poorly chosen because these persons are not blind, many of them are unaware that they have anything abnormal with their vision. However, if they compare such colors as moss green, snuff brown, dark purple, and dark grey, all these color may look more or less the same. Small deviations from normal affect only some specific working conditions. That is why color vision is examined at school before students get advice in career planning.

The screening examination uses pseudoisochromatic plates. Most commonly used test is called Ishihara's test. Screening tests are very sensitive and detect even minor deviations from normal color perception. They do not measure the degree of deviation. For the diagnosis of deviant color perception another test is necessary, a quantitative test in form of small caps with color surfaces in all colors of the spectrum. The diagnosis of color deficiency should never be based on a screening test. If a child seems to have any confusion with colors, color vision should be examined carefully. It can be started with clear basic colors to teach the concepts similar/different in relation to colors, after which quantitative testing is possible. Young children may train for the quantitative test by playing the Color Vision Game. Major color vision deficiencies are revealed already in this game but the diagnose requires proper measurement using pigment tests.

Binocular vision and three dimensional vision: We have two eyes but see only one picture, image. Visual information coming from the two eyes is fused into one image in the visual cortex. Not all normally sighted have binocular vision. They do not use both eyes simultaneously, together. Some persons look alternatingly with their right or left eye. They are usually unaware that they use their eyes separately. It does not disturb them.

Stereovision or three dimensional vision means that we have depth perception in near vision. When we look far away we have another kind of depth perception. We pay attention to the relative size of objects and which object is partially hidden behind another object. The speed of movement with which an object seems to move when we move our head or move around (called parallax) gives us clues on the distance. Therefore persons who do not have stereovision can still assess depth.

Dominant eye: Dominant or leading eye is the eye that we use when we look very carefully at near or at far and can use only one eye. Even when both eyes are used simultaneously one of the eyes is more dominant than the other. We have hand, foot, and eye preference.

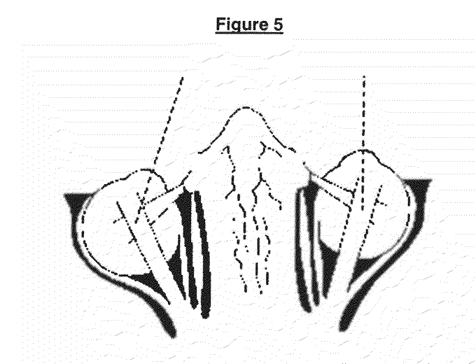

Eye motility and its disturbances: Eye movements are usually well controlled. The eyes look at the same object. Eyes turn because of the function of six eye muscles. If one of the eye muscles is paralysed, the eye turns in an abnormal position, the person sees double images (FIG. 5)

If an eye muscle is not functioning properly the person sees double when trying to look in the direction where the muscle should function. When the eyes are turned in the opposite direction the double image is fused again. The eye with the disturbed motility is covered until the muscle function returns to normal.

Sometimes there is no disturbance in the muscles themselves but the command to turn eyes in a certain direction is not handled normally because of changes in brain function.

Variation in the nature of visual disability: Different visual functions may become impaired independent of each other. Therefore there are many different types of visual impairement and disability. Sometimes a visually impaired person seems to function in a very confusing way. One moment (s)he seems to function like a normally sighted person and in the next moment like a blind person. A visually impaired person seldom pretends to see less than what (s)he actually sees.

One reason for variation in visual behavior might be changes in illumination. Another may be that (s)he knows the surroundings so there is no difficulty in orientation. Normally sighted persons move about the same way at home in the dark. They move confidently and securely as long there is nothing unexpected in their way. If somebody leaves an object on the usual path they may trip over it. In the very same way a visually impaired person needs only a few visual cues in a well-known place in order to be able to move freely.

If it is difficult to understand how a visually impaired person sees it is quite proper to ask him/her about his/her vision. Most visually impaired people are able to describe the nature of their impairment so well that it is possible to understand their situation better. Some persons say that they have only 10% vision left. Such a number does not describe the degree of visual impairment. The person may be able to move freely relying on his/her vision or may function like a nearly blind. That number (10%) usually means that his/her visual acuity is 20/200 (6/60 or 0.1) and it describes only one of many visual functions.

If the loss of visual functioning is caused by brain damage, the behavior of the person may look even more perplexing than when the loss is caused by changes in the eyes. In the higher visual functions, perceptual functions, small specific areas of the brain cortex are responsible for specific perceptions. If such an area with specific function is damaged, the corresponding function is either weak or completely lost. Thus an otherwise normally sighted person may not recognize people, not even close relatives. (S)he sees faces but cannot connect the visual information with pictures of faces in his/her memory.

There can be an isolated loss of motion perception, so that the person cannot tell whether a car is moving or not, or in milder cases, may perceive some movement but not how fast the car may be approaching. Color perception may be disturbed. Recognition of geometric forms may be lost and thus learning letters and numbers may be impossible.

The structure of egocentric space may be lost and thus concepts like ‘on the right’, ‘on the left’, ‘in the middle’, ‘next’, may be difficult. Also drawing of simple pictures or even copying pictures of angles may be impossible.

It is important that these children/adult persons are not diagnosed as intellectually disabled if they have other functions where they function normally. An uneven profile of functions should always lead to a thorough assessment of all cognitive visual functions and auditory perception. Children with loss of recognition of facial features or facial expressions are sometimes diagnosed as autistic, which is a tragic error and may negatively affect the child's future.

In one embodiment of the invention, the extrasynaptic GABA_{A} receptor antagonist of the present invention is selected from the group consisting of SR-95531 (Gabazine), pentylenetetrazole, bicuculline, bilobalide, ginkgolide B, picrotoxin, RO-4882224, RO-4938581, αSIA, and RG-1662; or a pharmaceutically acceptable salt thereof. The scope of the above compounds include the base compound of any pharmaceutically acceptable salt thereof.

SR-95531, also known as gabazine has the following structure (shown below as the hydrobromide salt):

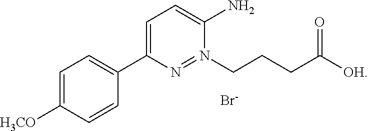

Another way of representing the structure of the hydrobromide salt of SR-95531 is the following;

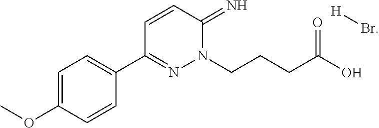

and is commercially available from Sigma-Aldrich and Tocris Bioscience. It's chemical name is: 6-Imino-3-(4-methoxyphenyl)-1(6H)-pyridazinebutanoic acid hydrobromide.

Pentylenetetrazole is also known chemically as: α,β-Cyclopentamethylenetetrazole, 1,5-Pentamethylenetetrazole, 6,7,8,9-Tetrahydro-5H-tetrazolo[1,5-a]azepine, and Metrazole. It has the chemical structure:

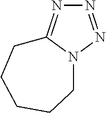

and is available commercially from Sigma-Aldrich.

(+)-Bicuculline has the following chemical structure:

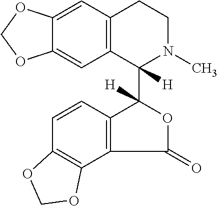 [SMILES: CN1CCc2cc3c(cc2[C@H]1[C@@H]1OC(=O)c2c1ccc1c2OCO1)OCO3]

and is available commercially from Sigma-Aldrich.

(−)-Bilobalida (from Ginkgo biloba leaves) has the following chemical structure:

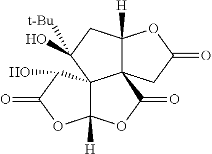 [SMILES: CC(C)(C)[C@]1(O)C[C@@H]2OC(=O)C[C@@]23C(=O)O[C@@H]2OC(=O)[C@H](O)[C@]213]

and is available commercially from Sigma-Aldrich.

Ginkolide B has the chemical name: (1R,3S,3aS,4R,6aR,7aR,7bR,8S,10aS,1-1R,11aR)-3-(1,1-Dimethylethyl)hexahydro-4,7b,11-trihydroxy-8-methyl-9H-1,7a-(epoxymethano)-1H,6aH-cyclopenta[c]furo[2,3-b]furo[3′,2′:3,4]cyclopenta[1,2-d]furan-5,9,12(4H)-trione. It has the following chemical structure:

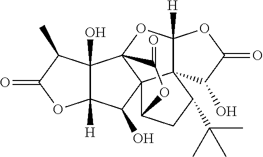

and is available commercially from Tocris Bioscience.

Picrotoxin, a 1:1 mixture of picrotoxinin and picrotin has the following chemical structure:

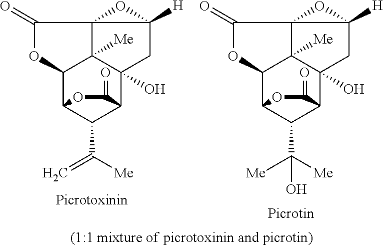

and is available commercially from Tocris Bioscience.

RO-4882224 has the following chemical structure:

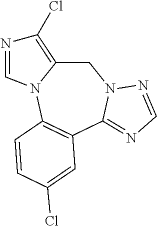 [SMILES: Clc1ccc2c(c1)-c1ncnn1Cc1c(Cl)ncn1-2]

and the chemical name: 3,10-Dichloro-9H-imidazo[1,5-a][1,2,4]triazolo[1,5-d][1,4]benzodiazepine.

RO-4938581 has the chemical name: 3-bromo-10-difluoromethyl-9H-imidazo[1,5-a][1,2,4]triazolo[1,5-d][1,4]benzodiazepine, and the following chemical structure:

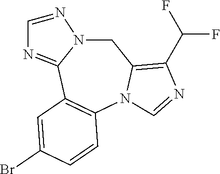 [SMILES: FC(F)c1ncn2c1Cn1ncnc1-c1cc(Br)ccc1-2]

The synthesis of both of the above two compounds (RO-4882224 and RO-4938581) is disclosed in: Henner Knust et al., “The discovery and unique pharmacological profile of RO4938581 and RO4882224 as potent and selective GABA_{A} α5 inverse agonists for the treatment of cognitive dysfunction”, Bioorganic & Medicinal Chemistry Letters, Volume 19, Issue 20, 15 Oct. 2009, Pages 5940-5944.

The compound α5IA has the IUPAC name: 5-methyl-3-[6-[(1-methyltriazol-4-yl)methoxy]-[1,2,4]triazolo[3,4-a]phthalazin-3-yl]-1,2-oxazole or 3-(5-methylisoxazol-3-yl)-6-[(1-methyl-1H-1,2,3-triazol-4-yl)methoxy][1,2,4]triazolo[3,4-a]phthalazine and the chemical structure:

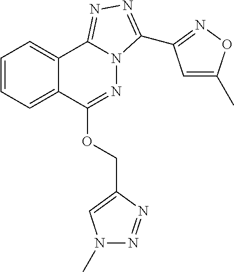 [SMILES: Cc1cc(-c2nnc3c4ccccc4c(OCc4cn(C)nn4)nn23)no1]

It is available from the following chemical vendors: ABI Chem, AKos Consulting & Solutions, and IS Chemical Technology.

RG-1662 is being developed in phase I clinical studies at Roche for the treatment of Alzheimer's type dementia and to improve cognition and adaptive behavior in adults with Down's syndrome.

In another embodiment, the extrasynaptic GABA_{A} receptor antagonist is a benzodiazepine site inverse agonist.

In another embodiment, the benzodiazepine site inverse agonist is selected from the group consisting of Ro 19-4603, Ro 15-4513, L-655,708, TB 21007, and MRK 016, all of which are commercially available from Tocris Bioscience.

Ro 15-4513 has the chemical name: 8-Azido-5,6-dihydro-5-methyl-6-oxo-4H-imidazo[1,5-a][1,4]benzodiazepine-3-carboxylic acid ethyl ester, and the chemical structure:

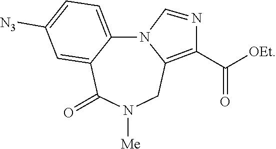 [SMILES: CCOC(=O)c1ncn2c1CN(C)C(=O)c1cc(N=[N+]=[N-])ccc1-2]

Ro 19-4603 has the chemical name: 5,6-Dihydro-5-methyl-6-oxo-4H-imidazo[1,5-a]thieno[2,3-f][1,4]diazepine-3-carboxylic acid 1,1-dimethylethyl ester, and the chemical structure:

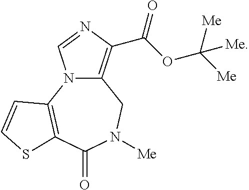 [SMILES: CN1Cc2c(C(=O)OC(C)(C)C)ncn2-c2ccsc2C1=O]

L-655,708 has the chemical name: 11,12,13,13a-Tetrahydro-7-methoxy-9-oxo-9H-imidazo[1,5-a]pyrrolo[2,1-c][1,4]benzodiazepine-1-carboxylic acid, ethyl ester, and the chemical structure:

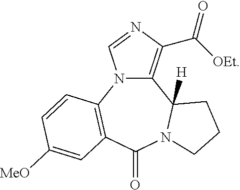 [SMILES: CCOC(=O)c1ncn2c1[C@@H]1CCCN1C(=O)c1cc(OC)ccc1-2]

TB 21007 has the chemical name: 6,7-Dihydro-3-[(2-hydroxyethyl)thio]-6,6-dimethyl-1-(2-thiazolyl)-benzo[c]thiophen-4(5H)-one, and the chemical structure:

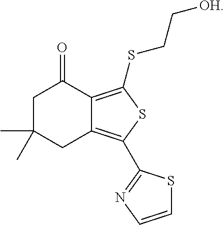 [SMILES: CC1(C)CC(=O)c2c(SCCO)sc(-c3nccs3)c2C1]

MRK 016 has the chemical name: 3-(1,1-Dimethylethyl)-7-(5-methyl-3-isoxazolyl)-2-[(1-methyl-1H-1,2,4-triazol-5-yl)methoxy]-pyrazolo[1,5-d][1,2,4]triazine, and the chemical structure:

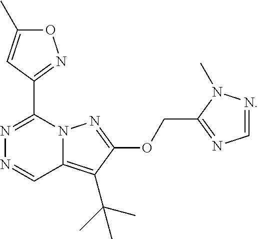 [SMILES: Cc1cc(-c2nncc3c(C(C)(C)C)c(OCc4ncnn4C)nn23)no1]

In another embodiment, the extrasynaptic GABA_{A} receptor antagonist of the present invention is SR-95531 (gabazine) or a pharmaceutically acceptable salt thereof.

In another embodiment, the visual acuity enhance in the present invention is measured by sweep vision evoked potential (sVEP).

In another embodiment, administration of the extrasynaptic GABA_{A} receptor antagonist compound enhances the receptive field profile of the retinal ganglion cells near the center of the receptive field.

In another embodiment, the subject in need of the visual enhancement in the present invention is one who has low or poor visual acuity resulting from a retinal disorder or retinal damage.

In another embodiment, the ocular condition resulting from the low/poor visual acuity in the present invention is selected from the group consisting of glaucoma, low-tension glaucoma, intraocular hypertension, wet and dry age related macular degeneration (AMD), geographic atrophy, macula edema, retinitis pigmentosa, Stargardt's disease cone dystrophy, and pattern dystrophy of the retinal pigmented epithelium, macular edema, retinal detachment and tears, retinal trauma, retinitis pigmentosa, retinal tumors and retinal diseases associated with said tumors, congenital hypertrophy of the retinal pigmented epithelium, acute posterior multifocal placoid pigment epitheliopathy, optic neuritis, acute retinal pigment epithelitis, diabetic retinopathy and optic neuropathies.

In another embodiment, the ocular condition resulting from the low/poor visual acuity in the present invention is selected from the group consisting of glaucoma, macular degeneration, wet and dry age related macular degeneration (AMD), geographic atrophy, and diabetic retinopathy.

In another embodiment, the administration of the GABA_{A} receptor antagonist enhances the receptive field profile of the retinal ganglion cells near the center of the receptive field.

The GABA_{A} receptor antagonists of the present invention can form salts which are also within the scope of this invention. Reference to a GABA_{A} receptor antagonist herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a GABA_{A} receptor antagonists contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the GABA_{A} receptor antagonists may be formed, for example, by reacting a such an antagonist with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The compounds of the present invention are administered intravitreally (e.g., through injection).

For intravitreal administration, the weight of the device (i.e., drug plus carrier/vehicle/excipinent) is typically 1 mg (which for example may be administered with a 22 G needle) and the drug load is normally 10-50%. The drug dose range for intravitreal administration is normally about 100-500 μg. However, the drug load can be stretched to 2-65%, i.e., a drug dose range of 20-650 μg can be used. However, the device weight may be 1.5 mg, and for this a drug dose range of 20-975 μg can be used.

Another way of intravitreal delivery is by injecting drug suspension formulation. For this, the dose range is 10-600 ug.

The intraocular implant of the present invention typically comprises a therapeutically effective amount of the presently disclosed GABA_{A} receptor antagonist (the therapeutic component; the active pharmaceutical ingredient (API)), and a drug release sustaining polymer component associated with the therapeutic compound. As used herein, an “intraocular implant” refers to a device or element that is structured, sized, or otherwise configured to be place in an eye. Intraocular implants are generally biocompatible with physiological conditions of an eye and do not cause adverse side effects. Intraocular implants may be place in an eye without disrupting vision of the eye.

The implant may be solid, semisolid, or viscoelastic. The drug release sustaining component is associated with the therapeutic component to sustain release of an amount of the therapeutic component into an eye in which the implant is placed.

The therapeutic component may be released from the implant by diffusion, erosion, dissolution or osmosis. The drug release sustaining component may comprise one or more biodegradable polymers or one or more non-biodegradable polymers. Examples of biodegradable polymers of the present implants may include poly-lactide-co-glycolide (PLGA and PLA), polyesters, poly(ortho ester), poly(phosphazine), poly(phosphate ester), polycaprolactone, natural polymers such as gelatin or collagen, or polymeric blends. The amount of the therapeutic component is released into the eye for a period of time greater than about one week after the implant is placed in the eye and is effective in reducing or treating an ocular condition.

In one embodiment, the intraocular implant comprises a therapeutic component and a biodegradable polymer matrix. The therapeutic component is associated with a biodegradable polymer matrix that degrades at a rate effective to sustain release of an amount of the therapeutic component from the implant effective to treat an ocular condition. The intraocular implant is biodegradable or bioerodible and provides a sustained release of the therapeutic component in an eye for extended periods of time, such as for more than one week, for example for about one month or more and up to 5 about six months or more. The implant may be configured to provide release of the therapeutic component in substantially one direction, or the implant may provide release of the therapeutic component from all surfaces of the implant.

The biodegradable polymer matrix of the foregoing implant may be a mixture of biodegradable polymers or the matrix may comprise a single type of biodegradable polymer. For example, the matrix may comprise a polymer selected from the group consisting of polylactides, poly(lactide-co-glycolides), polycaprolactones, and combinations thereof.

In another embodiment, the intraocular implant comprises the therapeutic component and a polymeric outer layer covering the therapeutic component. The polymeric outer layer includes one or more orifices or openings or holes that are effective to allow a liquid to pass into the implant, and to allow the therapeutic component to pass out of the implant.

The therapeutic component is provided in a core or interior portion of the implant, and the polymeric outer layer covers or coats the core. The polymeric outer layer may include one or more non-biodegradable portions. The implant can provide an extended release of the therapeutic component for more than about two months, and for more than about one year, and even for more than about five or about ten years. One example of such a polymeric outer layer covering is disclosed in U.S. Pat. No. 6,331,313.

In one embodiment, the present implant provides a sustained or controlled delivery of the therapeutic component at a maintained level despite the rapid elimination of the therapeutic component from the eye. For example, the present implant is capable of delivering therapeutically effective amounts of the therapeutic component for a period of at least about 30 days to about a year despite the short intraocular half-lives that may be associated with the therapeutic component. Plasma levels of the therapeutic component obtained after implantation may be extremely low, thereby reducing issues or risks of systemic toxicity. The controlled delivery of the therapeutic component from the present implants would permit the therapeutic component to be administered into an eye with reduced toxicity or deterioration of the blood-aqueous and blood-retinal barriers, which may be associated with intraocular injection of liquid formulations containing the therapeutic component.

A method of making the present implant involves combining or mixing the therapeutic component with a biodegradable polymer or polymers. The mixture may then be extruded or compressed to form a single composition. The single composition may then be processed to form individual implants suitable for placement in an eye of a patient.

Another method of making the present implant involves providing a polymeric coating around a core portion containing the therapeutic component, wherein the polymeric coating has one or more holes. The implant may be placed in an ocular region to treat a variety of ocular conditions, such as treating the conditions disclosed herein.

The daily dose may be administered as single dose or in divided doses and, in addition, the upper limit can also be exceeded when this is found to be indicated.

### Assays

### SR95531 Pharmacokinetics Methods

### Study Design:

One group of 12 male Dutch-belted rabbits received a single intravitreal injection of 50 μL of formulated SR95531 (360 μM, nominal concentration) in each eye on Study Day 1. Two rabbits were sacrificed at each of the six post-injection collection time-points (i.e., 1, 4, 10, 24, 48 and 168 hours).

### Sample Collections:

Whole blood was collected from the central ear artery of unanesthetized animals into K3-EDTA tubes and centrifuged at approximately 3,000 rpm for 10 minutes, under refrigeration.

Plasma samples stored frozen at approximately −70±15 C until shipment (on dry ice) to JCL Bioassay USA, Inc. for bioanalysis. Ocular Tissues (vitreous humor, retina and choroid) samples were collected from both eyes of each animal immediately after euthanasia, weighed and stored frozen at approximately −70±15 C until shipment (on dry ice) to the bioanalytical laboratory (JCL Bioassay USA, Inc.).

### Analytical Method:

Plasma and tissue samples were analyzed by High-Performance Liquid Chromatography/Tandem Mass Spectrometry. The analysis was run on a Shimadzu Nexera UHPLC system coupled to an AB Sciex Triple Quad 5500 operated in the positive electrospray mode. The calibration range was as follows: for tissues (vitreous humor, retina, choroid), the calibration standards bracketing the samples ranged between 5 pg/mL and 10 ng/mL; for plasma, the calibration standards bracketing the samples ranged between 0.2 and 400 ng/mL. Results below the lower limit of quantitation were reported as BLQ.

### Pharmacokinetic Analysis:

Pharmacokinetic (PK) analysis was performed on each composite mean concentration-time curve. Non-compartmental pharmacokinetic analyses were performed using WinNonlin® software, version 5.3. (Pharsight Corporation, Mountain View, Calif.) and model 201 (used for bolus IV input) for vitreous humor and model 200 (used for extravascular input) for the retina, choroid and plasma.

### Optomotor Measurements:

Rabbits were placed on a platform in the center of an arena consisting of 4 computer monitors forming the faces of an open cube that displayed sine wave gratings as a virtual cylinder. Each animal's daily maximal threshold was generated by incrementally increasing the spatial frequency until the rabbits no longer tracked the stimulus as described previously (Douglas et al., 2005; Prusky et al., 2004). Following 14 days of acclimation measurements, 5 uM intravitreal dose (50 uL of 120 uM) of SR95531 was injected intravitreally and measurements were continued for up to 5 days. It is worthy to note that initially both saline and drug injected eyes resulted in drop in acuity threshold presumably due to discomfort caused by the injection. 24 hours post-injection, the effects of the drug were clearly visible. These experiments were conducted in a blind method where the experimenter was not aware as to which eye received the drug or saline.

Sweep Vision Evoked Potential (sVEP) Measurements.
sVEP is an indirect measure of visual acuity and is highly correlated with snellen acuity in humans (Ridder 2004). sVEP is a tool that is often used to assess visual function in human infants and animal models since these subjects can't read a Snellen chart or communicate with the test administrator (Norcia et al., 1985; Guire et al., 1999). Sweep VEP (sVEP) threshold is measured at the point where the signal meets the noise. Sweep VEP (sVEP) measurements were made from awake Dutch-belted rabbits using a spatial frequency range from 0.3 to 5 cycles per degree at 80% contrast using the Power-Diva system. Following control recordings, an intravitreal injection of 1, 5, 15 and 50 uM (intravitreal concentration) SR95531 were made and the recording was repeated for up to 14 days post injection (see figure for more details). 50 uL Intravitreal injections of concentrated dose (24 fold to account for rabbit vitreal dilution) of the drug were made with a 30 guage hyperdermic needle and a Hamilton syringe.

### Evaluation of GABA Levels in the Vitreous Humor:

Eight Dutch belted rabbits were used in this study. One eye was injected with 3 mM intravitreal concentration (50 uL of 72 mM) of NMDA while the contralateral eye remained naïve. Two weeks after intravitreal dosing the animals were euthanized in two groups, the eyes in one group (4 rabbits) were enucleated during the day (11 am-1 pm) while the eyes in the other group (4 rabbits) were enucleated at night (11 pm-1 am). Immediately following enucleation, the anterior portion of the eye was removed and radial cuts were made to the schlera to flatten the eyecup. The vitreous was gently removed, weighed and placed in a 3 mL mixture of acetonitrile and water (ratio of 3:1) and stored at −80 C. Samples were thawed, and vortexed vigorously for approximately 1 minute and 200 μL of the solution was transferred to a clean tube where 25 μL of internal standard solution (GABA-d6 radio-labeled GABA in acetonitrile) was added, vortexed, centrifuged briefly, and a portion injected onto the Liquid Chromotography Mass Spectrometer (LCMS) for quantification. GABA standard curves were generated using serial dilutions which resulted in GABA concentration ranging from 10 ng/mL to 20 ug/mL and an acceptance criteria was set such that 75% of all standards were within 70-130% of the nominal value.

What is claimed is:

1. A method of enhancing visual function in a subject, comprising intravitreally administering to the subject in need of such enhancement, a therapeutically effective amount of a compound that is an extrasynaptic GABA_{A} receptor antagonist.

2. The method of claim 1, wherein the visual function is selected from the group consisting of visual acuity, visual field, contrast sensitivity, visual adaptation to differerent luminance levels, color vision, binocular and three dimensional vision

3. The method of claim 2, wherein the visual function is visual acuity.

4. The method of claim 1, wherein the compound is SR-95531 (Gabazine), or a compound selected from the group consisting of pentylenetetrazole, bicuculline, bilobalida, ginkgolide B, picrotoxin, RO-4882224, RO-4938591, α5IA, and RG-1662; or a pharmaceutically acceptable salt thereof.

5. The method of claim 1, wherein the compound is a benzodiazepine site inverse agonist.

6. The method of claim 5, wherein the benzodiazepine site inverse agonist is selected from the group consisting of: Ro 19-4603, Ro 15-4513, L-655,708, TB 21007, and MRK 016; or a pharmaceutically acceptable salt thereof.

7. The method of claim 1, wherein the subject in need of such enhancement is one who has low/poor visual function resulting from a retinal disorder or retinal damage.

8. The method of claim 3, wherein said visual acuity is measured by sweep vision evoked potential (sVEP).

9. The method of claim 1, wherein administration of the compound enhances the receptive field profile of the retinal ganglion cells near the center of the receptive field.

10. A method of treating an ocular condition resulting from low/poor visual function in a subject, comprising intravitreally administering to said subject in need of such treatment, a therapeutically effective amount of a compound that is an extrasynaptic GABA_{A} receptor antagonist.

11. The method of claim 10, wherein said ocular condition is selected from the group consisting of glaucoma, low-tension glaucoma, intraocular hypertension, wet and dry age related macular degeneration (AMD), geographic atrophy, macula edema, Stargardt's disease cone dystrophy, and pattern dystrophy of the retinal pigmented epithelium, macular edema, retinal detachment and tears, retinal trauma, retinitis pigmentosa, retinal tumors and retinal diseases associated with said tumors, congenital hypertrophy of the retinal pigmented epithelium, acute posterior multifocal placoid pigment epitheliopathy, optic neuritis, acute retinal pigment epithelitis, diabetic retinopathy and optic neuropathies.

12. The method of claim 10, wherein the compound is SR-95531 (Gabazine), or a compound selected from the group consisting of pentylenetetrazole, bicuculline, bilobalida, ginkgolide B, picrotoxin, RO-4882224, RO-4938591, α5IA, and RG-1662, or a pharmaceutically acceptable salt thereof.

13. The method of claim 10, wherein the compound is a benzodiazepine site inverse agonist.

14. The method of claim 13, wherein the benzodiazepine site inverse agonist is selected from the group Ro 19-4603, Ro 15-4513, L-655,708, TB 21007, and MRK 016; or a pharamaceutially acceptable salt thereof.

15. The method of claim 12, wherein the compound is SR-95531 or a pharmaceutically acceptable salt thereof.

16. An ocular implant comprising a therapeutically effective amount of a compound that is an extrasynaptic GABA_{A} receptor antagonist.

17. The implant of claim 16, wherein the compound is SR-95531 (Gabazine), or a compound selected from the group consisting of pentylenetetrazole, bicuculline, bilobalida, ginkgolide B, picrotoxin, RO-4882224, RO-4938591, α5IA, and RG-1662; or a pharmaceutically acceptable salt thereof.

18. The implant of claim 16, wherein the compound is a benzodiazepine site inverse agonist.

19. The implant of claim 16, wherein the benzodiazepine site inverse agonist is selected from the group Ro 19-4603, Ro 15-4513, L-655,708, TB 21007, and MRK 016; or a pharmaceutically acceptable salt thereof.

20. The implant of claim 17, wherein the compound is SR-95531 or a pharmaceutically acceptable salt thereof.
